(19) United States
(12) Patent Application Publication
(10) Pub. No.: US 2025/0057608 A1
(43) Pub. Date: Feb. 20, 2025

(54) SMART CONNECTORS FOR ROBOTIC TOOLS

(71) Applicant: Alcon Inc., Fribourg (CH)

(72) Inventor: Steven T. Charles, Memphis, TN (US)

(21) Appl. No.: 18/796,670

(22) Filed: Aug. 7, 2024

Related U.S. Application Data
(60) Provisional application No. 63/518,363, filed on Aug. 17, 2023.

(51) Int. Cl. A61B 34/30; A61B 17/00; A61B 90/00; A61B 90/98; A61F 9/007; B25J 15/00

(52) U.S. Cl. CPC A61B 34/30; A61B 90/98; B25J 15/0019; A61B 2017/00725; A61B 2090/061; A61F 9/007

(57) ABSTRACT

Systems and methods for performing robotic surgery are provided. The system may include a robotic manipulator having a working end coupled to a base by a plurality of joints and links, a surgical tool having a tool portion and a connector portion coupled to the working end of the robotic manipulator, and a control system having a tool interface operable to communicate with the surgical tool, a robotic manipulator interface communicatively coupled to the robotic manipulator, and a processor coupled to the tool interface and the robotic manipulator interface. The processor may be configured to receive data from the surgical tool and control the robotic manipulator based on the data received from the surgical tool. The method may include receiving data from a surgical tool connected to a working end of a robotic manipulator and controlling the robotic manipulator based on the data received from the surgical tool.

### TECHNICAL FIELD

The present disclosure relates generally to robotic tools and more particularly, but not by way of limitation, to smart connectors for robotic tools.

### BACKGROUND

This section provides background information to facilitate a better understanding of the various aspects of the disclosure. It should be understood that the statements in this section of this document are to be read in this light, and not as admissions of prior art.

Many ocular surgical procedures have the potential to utilize robotics in order to accurately perform surgery. However, manual data entry of surgical tool data into a surgical system can be error prone and fails to adequately address the need to efficiently perform surgeries and to accurately determine, for example, gravity compensation and collision avoidance of surgical tools in an operating environment.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In certain embodiments, the disclosure relates to a method that includes, without limitation, receiving data from a surgical tool connected to a working end of a robotic manipulator and controlling the robotic manipulator based, at least in part, on the data received from the surgical tool.

In certain embodiments, the disclosure relates to a system that may include, without limitation, a robotic manipulator having a working end coupled to a base by a plurality of joints and a plurality of links, a surgical tool having a tool portion and a connector portion coupled to the working end of the robotic manipulator, and a control system. The control system may include, without limitation, a tool interface operable to communicate with the surgical tool, a robotic manipulator interface communicatively coupled to the robotic manipulator, and a processor coupled to the tool interface and the robotic manipulator interface. The processor may be configured to receive data from the surgical tool and control the robotic manipulator based, at least in part, on the data received from the surgical tool.

### BRIEF DESCRIPTION OF THE DRAWINGS

A more complete understanding of the subject matter of the present disclosure may be obtained by reference to the following Detailed Description when taken in conjunction with the accompanying drawings wherein:

FIG. 1 illustrates a perspective view showing a surgery being performed in an operating environment with a surgical system, according to embodiments of the present disclosure.

FIG. 2 illustrates a perspective view of a robotic manipulator for use with the surgical system of FIG. 1, according to embodiments of the present disclosure.

FIG. 3 illustrates a tool assembly at a working end of the robotic manipulator of FIG. 2, according to embodiments of the present disclosure.

FIGS. 4A, 4B illustrate schematics of various embodiments of a tool assembly that is removable from a robotic manipulator of FIG. 2, according to embodiments of the present disclosure.

FIG. 5A illustrates a robotic manipulator connection interface, according to embodiments of the present disclosure.

FIGS. 5B, 5C illustrate various embodiments of connection interface of a tool connector, according to embodiments of the present disclosure.

FIG. 6 illustrates a block diagram schematically illustrating components of a control system of a robotic surgical system, according to embodiments of the present disclosure.

FIG. 7 illustrates a flow diagram illustrating a method of controlling a robotic manipulator, according to embodiments of the present disclosure.

FIG. 8 illustrates a flow diagram of a method for applying a torque value to a joint of a robotic manipulator, according to embodiments of the present disclosure.

FIG. 9 illustrates a flow diagram of a method for avoiding collision with obstacles in an operational volume of a robotic manipulator, according to embodiments of the present disclosure.

FIG. 10 illustrates a flow diagram of a method for calibrating operation of a surgical tool, according to embodiments of the present disclosure.

FIG. 11 illustrates a flow diagram of a method for controlling a surgical tool and a robotic manipulator based on identification data of the surgical tool, according to embodiments of the present disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Many ocular surgical procedures have the potential to utilize robotics in order to accurately perform surgery. However, manual data entry of surgical tool data into surgical systems can be error prone and, additionally, fails to adequately address the need to efficiently perform surgeries and to accurately determine gravity compensation and collision avoidance of surgical tools in an operating environment. This disclosure presents various embodiments that allow for convenient tool exchange using a connector, that in some embodiments can be universal, while also allowing for the transfer of data seamlessly to and from a control system and/or a robotic manipulator from a surgical tool.

As used herein, the term “surgical tool” may refer to any surgical tool or device for performing a surgical procedure. For example, the term “surgical tool” may refer to a surgical tool, such as a phacoemulsification tool, a vitrectomy tool, a passive tool (such as a blade, etc.), an actuated tool (such as a drill, a saw, a cutter, etc.), an optical tool (such as a laser, a light, etc.), or any other tools or devices used in an ophthalmic or intraocular operating room, as known to one of ordinary skill in the art.

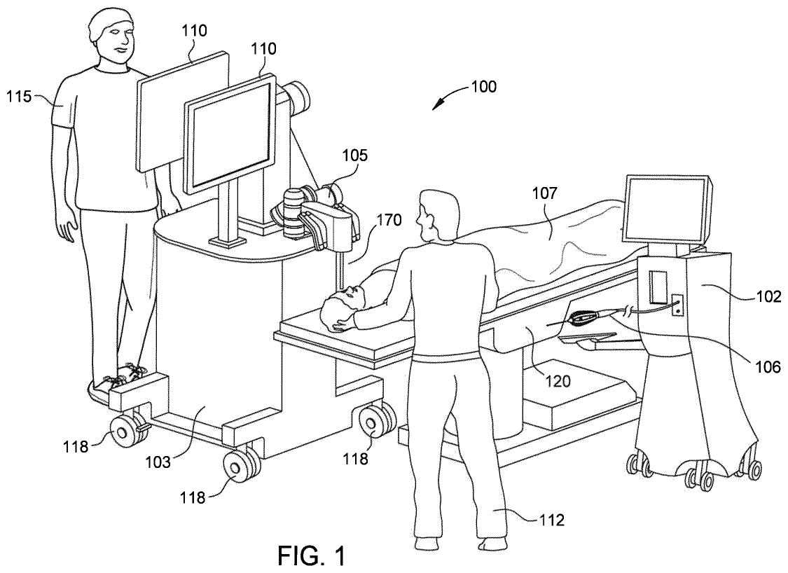

FIG. 1 illustrates a perspective view showing a surgery being performed in an operating environment with surgical system 100 according to aspects of this disclosure. The system 100 includes a surgical console 102 and a robotic manipulator 105, such as a serial manipulator, a parallel manipulator, a hybrid serial-parallel manipulator, a manipulator arm, a robotic arm, etc. The surgical console 102 may include, or be operably coupled to (e.g., physically or wirelessly), one or more modules, systems, devices, and/or surgical tools for performing one or more surgical procedures. For example, in certain embodiments, the surgical console 102 may include one or more ports for coupling a surgical tool 106 to an internal fluid source, vacuum source, and/or driver. In certain embodiments, the surgical console 102 may be in physical or wireless communication with the robotic manipulator 105.

The robotic manipulator 105 is operable to hold, move, and/or insert a tool assembly into and within an interior and/or exterior volume of an eye of a medical patient 107. Although it is currently contemplated that the medical patient will most frequently be a human patient, in veterinary applications the “patient” can be a non-human animal, and the term as it appears in this disclosure should be read to encompass either possibility.

The robotic manipulator 105 may be supported by any suitable device or system within an operating environment. In the example of FIG. 1, the robotic manipulator 105 is supported by a cart 103. The cart 103 can itself be fixed or movable with respect to a structure for supporting the medical patient 107. FIG. 1 depicts an installation in which the cart 103 is supported on wheels 118 that allow the cart 103 to be rolled into position with respect to a patient support table 120, which is illustrated as fixed to the floor of a surgical suite in which the procedure is performed. In other embodiments, however, the robotic manipulator 105 may be supported by the surgical console 102 or another supporting structure within the operating environment. In particular embodiments, the robotic manipulator 105 might, for example, be supported on a movable cart (e.g., a device, case, or supply cart), the floor, ceiling, or a wall of a surgical suite or another room in which the equipment is located, a bed or a similar structure on which the patient is placed for treatment, or any other suitable location. In certain embodiments, the robotic manipulator 105 is movably attached to a track or rail upon which the robotic manipulator 105 may translate. Such track or rail may be disposed on, for example, the surgical console 102, the cart 103, a movable cart, the floor, ceiling, or a wall of a surgical suite or another room.

The system 100 may further include one or more display monitors 110, or other visualization devices such as augmented reality (AR) or virtual reality (VR) headsets and the like, for delivering information and images to medical personnel in the course of a surgery. FIG. 1 illustrates a system with two display monitors 110 disposed on the cart 103 for use, for example, by a surgeon 112 and an assistant 115. The display monitors 110 can be communicatively coupled with the surgical console 102, a visualization system within the operating environment, and/or the tool assembly supported by the robotic manipulator 105. In some embodiments, the display monitors 110 can receive information (e.g., surgical parameters) and/or images from the surgical console 102 and tool assembly, respectively, and display the information and images on the display monitors 110. The surgical console 102 can also send signals to the display monitors 110 for performing operations (e.g., starting and stopping video recording).

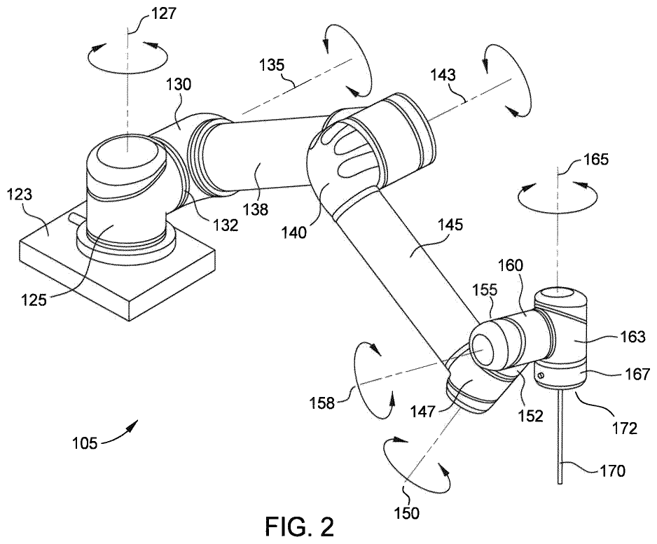

FIG. 2 illustrates a perspective view of robotic manipulator 105 for use with surgical system 100 of FIG. 1 according to certain aspects of this disclosure. With respect to FIG. 2, elements of the robotic manipulator 105 are illustrated in more detail. As shown, the robotic manipulator 105 includes a support base 123 which may, in certain embodiments, be configured to mount to the cart 103. Alternatively, as described above, the support base 123 may be mounted to another fixture within an operating environment, or may be a standalone component. The robotic manipulator further includes a first manipulator joint 125 that is rotatable with respect to the support base 123 around a first rotation axis 127.

A second manipulator joint 130 is fixed to the first manipulator joint 125 via a first link 132. The second manipulator joint 130 is rotatable with respect to the first manipulator joint 125 and the first link 132 around a second rotation axis 135. In certain embodiments, the second rotation axis 135 is perpendicular to the first rotation axis 127.

A second link 138 attaches a third manipulator joint 140 to the second manipulator joint 130. The third manipulator joint 140 can rotate with respect to the second link 138 around a third rotation axis 143. In certain embodiments, the third rotation axis 143 is parallel with the second rotation axis 135.

A third link 145 extends between the third manipulator joint 140 and a fourth manipulator joint 147. The fourth manipulator joint 147 rotates with respect to the third link 145 around a fourth rotation axis 150. In certain embodiments, the fourth rotation axis 150 is parallel with the second rotation axis 135.

A fourth link 152 joins the fourth manipulator joint 147 to a fifth manipulator joint 155. The fifth manipulator joint 155 rotates with respect to the fourth link 152 around a fifth rotation axis 158. In certain embodiments, the fifth rotation axis 158 is perpendicular to the first rotation axis 127.

A fifth link 160 attached to the fifth manipulator joint 155 carries a sixth manipulator joint 163, which rotates with respect to the fifth link 160 around a sixth rotation axis 165. In certain embodiments, the sixth rotation axis 165 is perpendicular to the fifth rotation axis 158. The sixth manipulator joint 163 carries sixth link 167. In this embodiment, a working element in the form of a surgical tool 170 extends from a working end 172 of the sixth link 167, in a direction parallel to the sixth rotation axis 165.

It should be noted that the configuration of the robotic manipulator 105 in FIG. 2 is illustrative of one embodiment, and the exact configuration of the robotic manipulator 105 can vary considerably in any given embodiment. In certain embodiments, the robotic manipulator 105 includes elements providing movement with at least six degrees of freedom (DOF) to facilitate effective minimally invasive surgery, or other minimally invasive procedures, at a target site within the patient's eye through a small incision on the eye's outer surface. However, in other embodiments, more or less than six DOF are readily envisioned.

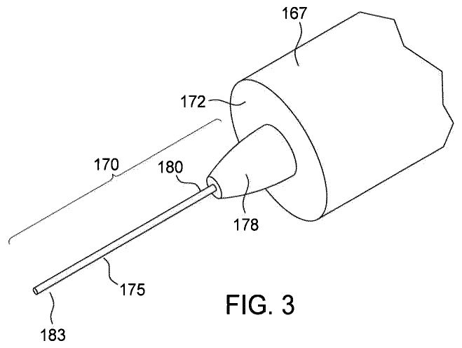

FIG. 3 illustrates surgical tool 170 at a working end of robotic manipulator 105 of FIG. 2 according to certain aspects of the disclosure. FIG. 3 depicts an embodiment of surgical tool 170 which can be coupled at the working end 172 of the sixth link 167. The surgical tool 170 includes a tool portion 175. The tool portion 175 is fixed to the working end 172 of the sixth link 167 via a mount 178 at a proximal end 180 of the tool portion 175. A distal end 183 of the tool portion 175 can include one or more working elements. In certain embodiments, the surgical tool 170 may be fixed permanently at the working end 172. However, in many cases it may be preferable that the surgical tool 170 be conveniently removable and/or replaceable at the working end 172.

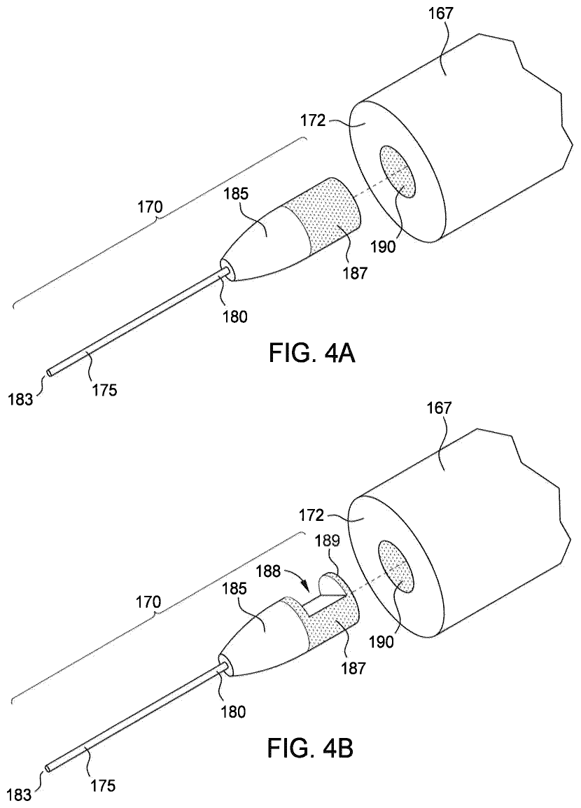

FIGS. 4A, 4B illustrate schematics of various embodiments of surgical tool 170 that is removable from robotic manipulator 105 of FIG. 2 according to certain aspects of the disclosure. Surgical tool 170 can be attached and detached at the working end 172 via a connector portion 185 at the proximal end 180 of the tool portion 175 of the surgical tool 170. For illustrative purposes, FIG. 4A shows the connector portion 185 as including press fit portion 187 on a proximal end thereof that interfaces with corresponding internal press fit portion within a recess 190 of the working end 172 of the last robotic manipulator link (e.g., the sixth link 167), so that the surgical tool 170 can be attached, mounted, held and secured, and detached or removed conveniently with respect to the robotic manipulator 105.

In certain embodiments illustrated in FIG. 4B, the connector portion 185 can be shaped in order to reduce the overall weight of the connector portion 185, and thus the overall weight of an assembly connected to the robotic manipulator 105. As shown in FIG. 4B, the proximal end of the connector portion 185 is depicted as a semicircle structure with press fit portion 187. The semicircle structure is formed by removal of a section 188, that is a semicircle, of the connector portion 185 that is to interface with the corresponding press fit portion within the recess 190 of the working end 172 of the last robotic manipulator link (the sixth link 167).

In some embodiments, the support 189 is omitted, and the proximal end of the connector portion 185 ends in a configuration formed by the removal of section 188 (e.g., a semicircle or half-moon configuration). While FIG. 4B illustrates a semicircle structure removed from the section 188, other shapes and forms are readily envisioned that can be removed from the section 188. Such shapes can include, for example, V-shaped grooves, U-shaped grooves, wave patterns, patterned or un-patterned holes, half and/or partial moon shapes, and combinations of the same and like.

Additionally, any tool portion 175 can be associated with connector portion 185, and can be configured appropriately so that the connector portion 185 attaches the surgical tool 170 at the working end 172 with sufficient security, while allowing for removal and replacement of the surgical tool 170 as desired. As such, the connector portion 185 may become a universal connection interface with the robotic manipulator 105. While FIGS. 4A-4B illustrate the connector portion 185 as having press fit portion 187, any type of temporary or semi-permanent connections are readily envisioned. For example, in certain embodiments, the connector portion 185 can include barbed or poly-barbed connectors, slip connectors, socket connectors, spigot connectors, push-fit connectors, twist and lock connectors, quick connect connectors, flared threaded connectors, compression connectors, crimp-type connectors, snap connectors, magnetic connectors, and combinations of the same and like. Other suitable retention assemblies for use with the connector portion 185 may include sliding and latching mechanisms of various configurations, or other assemblies for retaining the surgical tool 170 on the robotic manipulator 105 via the connector portion 185, as appropriate in any particular application.

A surgical tool 170 that is removable with respect to the robotic manipulator 105 can allow for the use of different types of surgical tools 170 for different procedures, for the removal and sterilization of a reusable surgical tool 170 between procedures, for the removal and disposal of a single-use, disposable surgical tool 170 after a completed procedure, and/or for other advantages as appropriate depending on the particular application. It may be preferable in many cases that the connector portion 185 allow removal and replacement of different surgical tools 170 at the working end 172 of the robotic manipulator 105, by a user of the system 100 manually and without the use of wrenches, screwdrivers, or other additional tools. In certain embodiments, the tool portion 175 is removable from the connector portion 185. In such embodiments, the connector portion 185 can act as a universal connector for various different tool portions 175. In other embodiments, the connector portion 185 can be an integrated part of the tool portion 175.

The connector portion 185 of the disclosure can act as a “smart” connector that is capable of transmitting data from the surgical tool 170 to a control system of, for example, the surgical console 102 or any other device within the system 100, as will be discussed in further detail below. In certain embodiments, the connector portion 185 can store and transmit information including, but not limited to, mass-related data, dimensional data, calibration data, tool identification data, and combinations of the same and like.

In certain embodiments, the mass-related data can include, without limitation, a mass of the surgical tool 170, a center of mass of the surgical tool 170 with respect to a surgical tool coordinate system, a moment of inertia of the surgical tool 170, a length of the surgical tool 170, and combinations of the same and like. In some embodiments, the dimensional data can include, for example, a length of the surgical tool 170, a first maximum offset from a centerline of the surgical tool 170 in a first dimension, a second maximum offset from a centerline of the surgical tool 170 in a second dimension, and a third maximum offset from a centerline of the surgical tool 170 in a third dimension. In certain embodiments, the first, second, and third dimensions correspond to an X-, Y-, and Z-plane of the surgical environment.

In certain embodiments, the calibration data can include the mass-related data, the dimensional data, and other data needed to calibrate the surgical tool 170 for use. Depending on the type of tool assembly, such data can include, without limitation, power requirements to operate the surgical tool 170, pressures required for pneumatic operations, light wavelengths or strengths for optical tools, and combinations of the same and like. In various embodiments, the tool identification data can include, without limitation, a serial number of the surgical tool 170, a description of the surgical tool 170, a name of the surgical tool 170, a type of tool assembly (e.g., optical, pneumatic, electric, etc.), and combinations of the same and like.

In certain embodiments, data related to the surgical tool 170 (e.g., mass-related data, dimensional data, calibration data, and tool identification data) can be stored via internal memory embedded within the connector portion 185. Such internal memory can include, without limitation, flash memory, random access memory (RAM), dynamic random access memory (DRAM), static random access memory (SRAM), read-only memory (ROM), programmable read-only memory (PROM), erasable programmable read-only memory (EPROM), electrically erasable programmable read-only memory (EEPROM), non-volatile memory, or various types of drives, such as, for example, a hard disk drive (HDD), a solid-state drive (SSD), or other non-transitory media. In certain embodiments, the data related to the surgical tool 170 can be stored in the form of a quick response (QR) code or a digital data tag. In certain embodiments, the data related to the surgical tool 170 can be stored in a radio frequency identification (RFID) chip or stored within near field communication (NFC) circuitry. In certain embodiments, the data related to the surgical tool 170 is stored via internal memory embedded within the connector portion 185 at a proximal end of the connector portion 185. In certain embodiments, the data related to the surgical tool 170 is stored via internal memory embedded within the connector portion 185 at a distal end of the connector portion 185.

In view of the aforementioned, the connector portion 185 can communicate with a control system of, for example, the surgical console 102 or any other device within the system 100, as will be discussed in further detail below. In such embodiments, the connector portion 185 can be operable to communicate via a wired connection, such as USB (Universal Serial Bus), Ethernet (IEEE 802.3), etc., and/or a wireless connection, such as Bluetooth, Bluetooth Low Energy (BLE), WiFi (IEEE 802.11), wireless PAN (Personal Area Network) (IEEE 802.15), NFC (Near Field Communication), RFID (Radio Frequency Identification), etc.

Some embodiments can include surgical tools 170 that can be driven or otherwise adjusted to articulate, or can be activated or otherwise engaged depending on the type of surgical tool 170 used. As such, the connector portion 185 can include connections within the connector portion 185 to operate the surgical tool 170 via optical connections, pneumatic connections, electrical connections, data connections, power connections, signal connections, and combinations of the same and like.

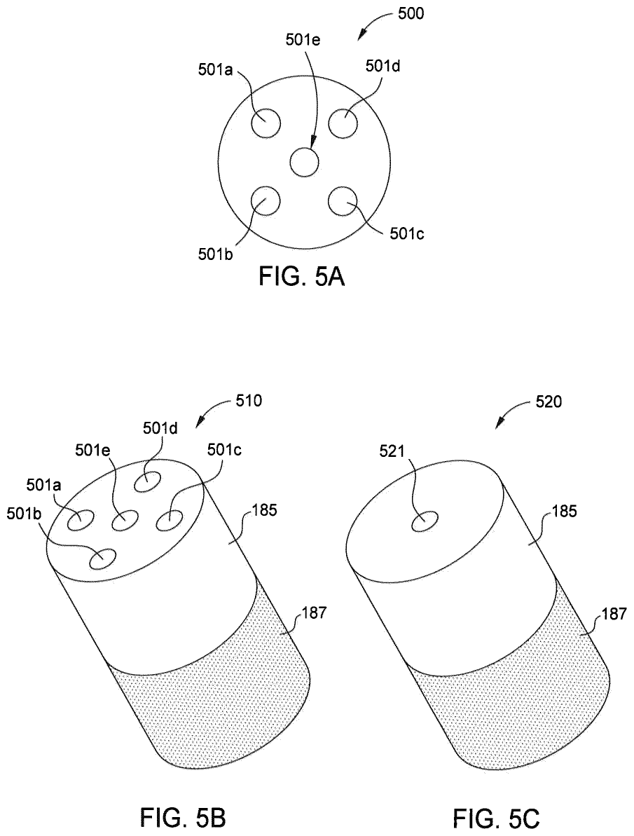

FIG. 5A illustrates robotic manipulator connection interface 500 according to certain aspects of the disclosure. The robotic manipulator connection interface 500 can be located at a terminal end within the recess 190 of the working end 172 to, for example, mate with a connection interface of the connector portion 185 once the surgical tool 170 has been attached via the connector portion 185 being received into the recess 190 (e.g., as depicted in FIGS. 4A-4B). In certain embodiments, the robotic manipulator connection interface 500 can include a plurality of connection points 501a, 501b, 501c, 501d, and 501e (collectively referred to as connection points 501). The connection points 501 provide, for example, various means for operating the surgical tool 170. The connection points 501 can provide, for example, optical, pneumatic, electrical, data, power, or signal connections, and various combinations of the same and like. The connection points 501 can provide some or all required connections to the surgical tool 170.

FIGS. 5B, 5C illustrate various embodiments of tool connection interface 510 and 520 of connector portion 185 according to certain aspects of the present disclosure. As shown in FIG. 5B, the tool connection interface 510 has a plurality of connection points 511a, 511b, 511c, 511d, and 511e (collectively connection points 511). In embodiments illustrated in FIG. 5B, the connection points 511a, 511b, 511c, 511d, and 511e align with, and interface/connect with 501a, 501b, 501c, 501d, and 501e, respectively, of the robotic manipulator connection interface 500. In some embodiments, one or more of the connection points 511 can be utilized to operate the surgical tool 170. Similar to connection points 501, the connection points 511 can provide one or more means for operating the surgical tool 170 (e.g., optical, pneumatic, electrical, data, power, or signal connections, control of suction lines, fibers for lasers and endo-illumination, and wires for radio frequency (diathermy), and combinations of the same and like). FIG. 5C illustrates embodiments where the tool connection interface 520 includes fewer connection points than the robotic manipulator connection interface 500. As shown in FIG. 5C, the tool connection interface 520 includes a connection point 521, which aligns with, and interfaces/connects to the connection point 501e of the robotic manipulator connection interface 500. It should be noted that the connection points 501, 511, and 521 are illustrative only, and that each of the robotic manipulator connection interface 500 and the tool connection interfaces 510 and 520 can include any number or arrangement of connection points.

FIGS. 5A-5C are for illustrative purposes only and are not intended to limit the scope of this disclosure. A person of ordinary skill in the art can readily envision various configuration patterns, numbers, and the like associated with the robotic manipulator connection interface 500 and the tool connection interfaces 510 and 520. It should further be noted that each connection point (e.g., connection points 501, 511, and 521) can align, interface, connect, or mate in various manners. For example, connection points 511 and 521 can align, interface, connect, or mate with connection points 501 via a contacted interface, a groove capable of receiving either connection point, latches, alignment of various elements such as washers, or combinations of the same and like as known to those of ordinary skill in the art.

The alignment, interfacing, connecting, or mating with connection points 501 and connection points 511 and 521 can also differ based on the type of temporary, or semi-permanent, connections of the connector portion 185 of the working end 172. Such types of alignment, interfacing, connecting, or mating can correspond with appropriate techniques when the connector portion 185 connects to the working end 172 using, for example, barbed or poly-barbed connectors, slip connectors, socket connectors, spigot connectors, push-fit connectors, twist and lock connectors, quick connect connectors, flared threaded connectors, compression connectors, crimp-type connectors, and combinations of the same and like. Additionally, alignment, interfacing, connecting, or mating can correspond with appropriate techniques when the connector portion 185 connects to the working end 172 using sliding and latching mechanisms of various configurations, and are readily envisioned by a person of ordinary skill in the art.

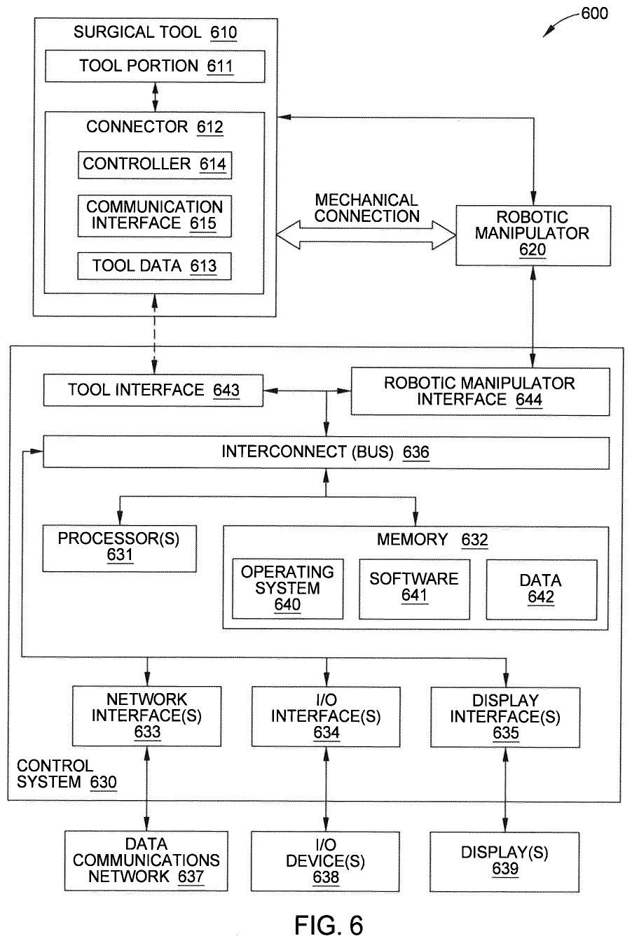

FIG. 6 illustrates a block diagram schematically illustrating components of control system 630 of robotic surgical system 600 according to certain aspects of the disclosure. The robotic surgical system 600 can include, for example, surgical tool 610, robotic manipulator 620, and control system 630. In certain embodiments, the surgical tool 610 can be any embodiment of the surgical tool 170 as depicted in FIGS. 1-4. Additionally, the robotic manipulator 620 can be representative of any of the embodiments of the robotic manipulator as detailed in FIGS. 1-2, and can be mechanically coupled to the surgical tool 610.

In certain embodiments, the control system 630 can be, for example, representative of a control system within, for example, the surgical console 102 of FIG. 1. In other embodiments, the control system 630 can be a standalone system residing within the system 100 of FIG. 1 for performing ophthalmic surgery. In various embodiments, the control system 630 can include processor(s) 631, memory 632, network interface(s) 633, input/output (I/O) interface(s) 634, and display interface(s) 635 coupled with an interconnect (bus) 636. The memory 632 can include operating system 640, software 641, and data 642. In general, the network interface(s) are communicatively coupled to a data communication network 637, the I/O interface(s) 634 are communicatively coupled to I/O device(s) 638, and the display interface(s) 635 are communicatively coupled to display(s) 639. The interconnect 636 transmits programming instructions and application data among the processor(s) 631, the network interface(s) 633, the I/O interface(s) 634, the display interface(s) 635, and/or the surgical tool 610 via a tool interface 643, and the robotic manipulator 620 via a robotic manipulator interface 644.

Interconnect 636 can include, for example, Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI Express (PCIe) bus, a serial advanced technology attachment (SATA) bus, and combinations of the same and like.

The processor(s) 631 may retrieve and store application data in data 642, process software 641, process operating system 640, as well as retrieve and execute instructions stored in the memory 632. The processor(s) 631 can represent a single central processing unit (CPU), multiple CPUs, a single CPU having multiple processing cores, a single graphics processing unit (GPU), multiple GPUs, a single GPU having multiple cores, and the like. In certain embodiments, the processor(s) 631 may include a microprocessor, controller, or any other suitable computing device, resource, or combination of hardware, software and/or encoded logic operable to execute, either alone or in conjunction with other components within the robotic surgical system 600.

Processor(s) 631 may include a single integrated circuit, such as a micro-processing device, or multiple integrated circuit devices and/or circuit boards working in cooperation to accomplish appropriate functionality. In addition, processor(s) 631 may execute computer programs or modules, such as operating system 640, software 641, and the like stored within memory 632.

In certain embodiments, memory 632 may be any form of volatile or non-volatile memory including, without limitation, magnetic media, optical media, RAM, ROM, flash memory, removable media, or any other suitable local or remote memory component or components. In particular embodiments, memory 632 may include RAM. This RAM may be volatile memory, where appropriate. Where appropriate, this RAM may be DRAM or SRAM. Moreover, where appropriate, this RAM may be single-ported or multi-ported RAM, or any other suitable type of RAM or memory. Memory 632 may include one or more memories 632, where appropriate.

Memory 632 may store any suitable data or information utilized by the control system 630, including software embedded in a computer readable medium, and/or encoded logic incorporated in hardware or otherwise stored (e.g., firmware). In particular embodiments, memory 632 may include main memory for storing instructions for processor(s) 631 to execute or data for processor(s) 631 to operate on.

Additionally, memory 632 may include mass storage for data or instructions. As an example and not by way of limitation, memory 632 may include a HDD, an SSD, flash memory, an optical disc, or a universal serial bus (USB) drive or a combination of two or more of these. Memory 632 may include removable or non-removable (or fixed) media, where appropriate. Memory 632 may be internal or external to the control system 630, where appropriate. In particular embodiments, memory 632 may be non-volatile, solid-state memory. In particular embodiments, memory 632 may include ROM. Where appropriate, this ROM may be mask-programmed ROM, PROM, EPROM, EEPROM, electrically alterable ROM (EAROM), or flash memory or a combination of two or more of these. Memory 632 may take any suitable physical form and may include any suitable number or type of storage.

In certain embodiments, memory 632 can include data 642 such as, for example, data related to the robotic manipulator 620, individual tool settings, operation settings, and the like. Additionally, in certain embodiments, memory 632 can include software 641, including for example, software and/or modules to determine gravity compensation requirements for surgical tool 610, software and/or modules to determine collision avoidance requirements for the surgical tool 610, software and/or modules to determine calibration parameters for the robotic manipulator 620 or the surgical tool 610, and the like.

Network interface(s) 633 is configured to transmit data to and from data communication network 637 using a wired connection, such as USB, Ethernet (IEEE 802.3), etc., a wireless connection, such as Bluetooth, BLE, WiFi (IEEE 802.11), wireless PAN (IEEE 802.15), NFC, RFID, etc., and/or a wireless cellular connection, such as code-division multiple access (CDMA), frequency-division multiple access (FDMA), time-division multiple access (TDMA), 4G, 4G long-term evolution (LTE), global systems for mobile (GSM), and 5G cellular networks, etc. For example, data communication network 637 may include a local area network (LAN) that is connected to a wide area network (WAN) through a router, the WAN may be connected to the Internet through an Internet Service Provider (ISP), and the like.

I/O interface(s) 634 are configured to transmit and/or receive data from I/O device(s) 638. I/O interface(s) 634 enable connectivity between processor(s) 631, memory 632, and I/O device(s) 638 by encoding data to be sent from processor(s) 631 or memory 632 to I/O device(s) 638, and decoding data received from I/O device(s) 638 for processor(s) 631 or memory 632. Generally, data may be sent over wired and/or wireless connections. For example, I/O interface(s) 634 may include one or more wired communications interfaces, such as USB, Ethernet (IEEE 802.3), etc., and/or one or more wireless communications interfaces, coupled to one or more antennas, such as Bluetooth, BLE, WiFi (IEEE 802.11), wireless PAN (IEEE 802.15), NFC, RFID, etc.

Generally, I/O device(s) 638 provide input to control the control system 630 and/or output from control system 630 to, for example, the robotic manipulator 620 and/or the surgical tool 610. I/O device(s) 638 are operably connected to control the control system 630 using a wired and/or wireless connection. I/O device(s) 638 may include a local processor coupled to a communication interface that is configured to communicate with control system 630 using the wired and/or wireless connection. In certain embodiments, I/O device(s) 638 may include, for example, a touch screen, keyboard, mouse, touch pad, joystick, or controls to manipulate the surgical tool 610 and/or the robotic manipulator 620. Display interface(s) 635 may be configured to transmit data (e.g., image data, data related to the surgical tool 610, or data related to the robotic manipulator 620) from control system 630 to display(s) 639. Display(s) 639 can include monitors, such as liquid crystal (LCD) displays, light-emitting diode (LED) displays, thin-film transistor (FTF) LCD displays, organic LED (OLED) displays, active-matrix organic LED (AMOLED) displays, and the like.

As illustrated in the embodiment of FIG. 6, the surgical tool 610 is communicatively/operatively and mechanically coupled to the robotic manipulator 620 (e.g., the robotic manipulator 105 or the like) via a connector 612, such that the robotic manipulator 620 can control and manipulate the surgical tool 610 (e.g., the surgical tool 170 or the like). In certain embodiments, the connector 612 can include any of the embodiments of the connector portion 185 as discussed relative to FIG. 4 and FIGS. 5B, 5C. Additionally, the robotic manipulator 620 can be communicatively/operatively and mechanically be coupled via a connector within the robotic manipulator 620, such as that described above relative to FIG. 4 and FIG. 5A.

As shown in FIG. 6, the connector 612 includes a controller 614 and a communication interface 615. In general, the controller 614 can include, without limitation, one or more microprocessors, CPUs, or any other suitable computing device, resource (e.g., an interconnect (bus) and/or memory), and/or combination of hardware, software and/or encoded logic operable to execute, either alone or in conjunction with other components within the surgical tool 610. In certain embodiments, the controller 614 can operate and/or manipulate a tool portion 611 of the surgical tool 610. In certain embodiments, the controller 614 provides a software and/or hardware (e.g., firmware) interface to the tool portion 611, enabling, for example, the control system 630 to directly or indirectly (e.g., via the tool interface 643) access hardware functions of the surgical tool 610. In various embodiments, the controller 614 communicates with the tool portion 611, such that when the control system 630 invokes a routine through the tool interface 643 to the connector 612, the controller 614 issues commands to the tool portion 611 of the surgical tool 610. In certain embodiments, the tool portion 611 can include, without limitation, a phacoemulsification tool, a vitrectomy tool, a laser tool, an optical tool, or any other tools or devices used in an ophthalmic or intraocular operating room, as known to one of ordinary skill in the art.

The communication interface 615 of the connector 612 is operable to communicate with the control system 630, or any other device within the system 600. In certain embodiments, the controller 614 is coupled to the communications interface 615 and is configured to send data (e.g., tool data 613) to the tool interface 643 of the control system 630. In certain embodiments, the communication interface 615 can be operable to communicate via a wired connection, such as USB, Ethernet (IEEE 802.3), etc., and/or a wireless connection, such as Bluetooth, BLE, WiFi (IEEE 802.11), wireless PAN (IEEE 802.15), NFC, RFID, etc. In some embodiments, the communication interface 615 can provide two way access and/or communication between the connector 612 and the tool interface 643. In various embodiments, the communication interface 615 can act in a similar manner with the robotic manipulator 620 via the robotic manipulator interface 644.

The connector 612 of the surgical tool 610 can further include tool data 613 as discussed relative to the connector 185 of FIG. 4. In certain embodiments, the tool data 613 can include, for example, mass-related data, dimensional data, calibration data, and tool identification data, such as that discussed with reference to FIG. 4. In certain embodiments, tool data 613 can include, without limitation, individual surgical tool 610 mass and moments to enable the robot manipulator 620 to render tools weightless (gravity compensation) in all poses of the robotic manipulator 620. Additional data can include, surgical tool 610 length relative to the working end, offsets for angulated surgical tools 610, external profile for collision avoidance, and inverse kinematics control laws. Data related to the surgical tool 610 actuation parameters and electrical or pneumatic actuation connectors can be incorporated in in the tool data 613. Additionally, tool data 613 can include identifying data, such as, individual operating parameters, a serial number, and a description and/or name of surgical tool 610.

The tool data 613 can be transferred to the tool interface 643 (e.g., via the communication interface 615) and stored in data 642 of memory 632 or other portions of memory 632. In certain embodiments, the connector 612 can transfer the tool data 613 to the tool interface 643 by way of a wired connection, such as USB, Ethernet (IEEE 802.3), etc., a wireless connection, such as Bluetooth, BLE, WiFi (IEEE 802.11), wireless PAN (IEEE 802.15), NFC, RFID, etc., or similar protocols utilizing the communication interface 615. In certain embodiments, the tool interface 643 can utilize the network interface(s) 633 in order to transfer tool data 613 from the connector 612 to the tool interface 643 such that the tool data 613 can be stored within data 642 or other portions of memory 632. In certain embodiments, the tool interface 643 can utilize the I/O interface(s) 634 in order to scan, for example, a QR code with I/O device(s) 638 such that tool data 613 is captured and stored within data 642 or other portions of memory 632.

As briefly described above, memory 632 can include software 641 that includes various modules and/or software components in order to determine various parameters required for operation. One such software component can include a gravity compensation module. With respect to gravity compensation, software 641 can pull tool data 613 from data 642 that was previously retrieved from the tool interface 643 from connector 612; however, in certain embodiments, software 641 can invoke the tool interface 643 to retrieve tool data 613 via connector 612 if the tool data 613 is not currently residing in data 642. Additionally, software 641 can pull data related to the robotic manipulator 620 from data 642. In certain embodiments, a plurality of surgical tools 610 can be utilized, as such, the software module can pull tool data 613 for each of the plurality of surgical tools 610. In some embodiments, a plurality of robotic manipulators 620 can be utilized, as such, the software module can pull data related to each robotic manipulator 620 from data 642.

Gravity compensation in all poses requires mass and moments data from each surgical tool 610. In general, when determining gravity compensation, software 641 will utilize mass-related data of the surgical tool 610 that can include, without limitation, a mass of the surgical tool 610, a center of mass of the surgical tool 610 with respect to a surgical tool coordinate system, a moment of inertia of surgical tool 610, a length of the surgical tool 610, and combinations of the same and like. Additionally, the software 641 can query data 642 to pull robotic manipulator data that can include, without limitation, number of joints of the robotic manipulator 620, length of each link between the joints of the robotic manipulator 620, weight of each link of the robotic manipulator 620, weight of motors in the robotic manipulator 620, and combinations of the same and like. In this manner, software 641 can calculate moments acting on each joint of the robotic manipulator 620. In response to determining moments for each joint of the robotic manipulator 620, the software 641 can send information to the robotic manipulator interface 644 to apply torque to each joint of the robotic manipulator 620. In certain embodiments, software 641 can calculate gravity compensation in real-time as to apply torque to each joint of the robotic manipulator 620 in real-time.

While gravity compensation was generally described with respect to a single surgical tool 610 and one robotic manipulator 620, a person of ordinary skill in the art can appreciate that such functionality can be applied to a plurality of surgical tools 610 and a plurality of robotic manipulators 620. In such instances, the software 641 can determine gravity compensation with respect to each robotic manipulator 620 and each surgical tool 610 such that moments acting on each joint of each robotic manipulator 620 is determined. In response to determining moments for each joint of each robotic manipulator 620, the software 641 can send information to the robotic manipulator interface 644 to apply torque to each joint of each robotic manipulator 620. In certain embodiments, software 641 can calculate gravity compensation in real-time as to apply torque to each joint of each robotic manipulator 620 in real-time.

Another software component can include a collision avoidance module. With respect to collision avoidance, software 641 can pull tool data 613 from data 642 that was previously retrieved from the tool interface 643 from connector 612; however, in certain embodiments, software 641 can invoke the tool interface 643 to retrieve tool data 613 via connector 612 if the tool data 613 is not currently residing in data 642. Additionally, software 641 can pull data related to the robotic manipulator 620 from data 642. In certain embodiments, a plurality of surgical tools 610 can be utilized, as such, the software module can pull tool data 613 for each of the plurality of surgical tools 610. In some embodiments, a plurality of robotic manipulators 620 can be utilized, as such, the software module can pull data related to each robotic manipulator 620 from data 642.

Collision avoidance, with for example, retina and lenses, requires information about length, offsets and outer envelope dimensions of intraocular portions of the surgical tool 610, as well as wide-angle visualization systems outside the eye. In general, when determining collision avoidance, software 641 can utilize dimensional data of the surgical tool 610 that can include, for example, a length of the surgical tool 610, a first maximum offset from a centerline of the surgical tool 610 in a first dimension, a second maximum offset from a centerline of the surgical tool 610 in a second dimension, and a third maximum offset from a centerline of the surgical tool 610 in a third dimension. Additionally, the software 641 can query data 642 to pull robotic manipulator data that can include, without limitation, number of joints of the robotic manipulator 620, length of each link between the joints of the robotic manipulator 620, location of each link and each joint of the robotic manipulator 620 in a first, second and third dimension (e.g., X-, Y-, and Z-planes), location of the robotic manipulator 620 relative to a surgical environment (e.g., the system 100 for performing ophthalmic surgery), a first maximum offset from a centerline of each link of the robotic manipulator 620 in a first dimension, a second maximum offset from a centerline of each link of the robotic manipulator 620 in a second dimension, and a third maximum offset from a centerline of each link of the robotic manipulator 620 in a third dimension, and combinations of the same and like. Furthermore, software 641 can pull surgical parameters from data 642 that can include location and dimensional data of the patient relative to the surgical tool 610 and the robotic manipulator 620, the type of surgical operation to be performed, eye curvature, size, shape, dimensions and the like of the patient, dimensions of a lens on the eye of the patient if lenses are used during surgical procedures, information related to wide angle visualization system parameters (contact and/or non-contact), and combinations of the same and like.

In this manner, software 641 can calculate a three-dimensional location of each link of the robotic manipulator 620 and the surgical tool 610 to determine whether or not the surgical tool 610 or robotic manipulator 620 will come into proximity to any undesired location (e.g., inadvertently puncturing an eye of the patient or a lens being worn by the patient). In certain embodiments, digital imaging can be utilized in determination of collision avoidance. In such embodiments, cameras and/or sensors can be a part of the surgical tool 610, a part of the robotic manipulator 620, or externally located such that the cameras and/or sensors are in a periphery of the surgical environment (e.g., the system 100 for performing ophthalmic surgery). In certain embodiments, the software 641 performs collision avoidance routines in real-time such that the surgical tool 610 and the robotic manipulator 620 are continuously being monitored to avoid potential collisions.

In certain embodiments, upon detection of a potential collision, software 641 can invoke the robotic manipulator interface 644 to stop or move the robotic manipulator 620 along a different trajectory before collision. Additionally, software 641 can include proximity alarms and/or alerts to indicate that a potential collision is imminent. In this manner, a surgeon (e.g., surgeon 112) can divert or stop the robotic manipulator 620 before collision. In some embodiments, the alarms and/or alerts can include, without limitation, visual or audible alarms and/or alerts. In certain embodiments, the surgeon can override the software 641 in cases where the surgeon would like to insert a portion of the surgical tool 610 into, for example, an eye of a patient.

While collision avoidance was generally described with respect to a single surgical tool 610 and one robotic manipulator 620, a person of ordinary skill in the art can appreciate that such functionality can be applied to a plurality of surgical tools 610 and a plurality of robotic manipulators 620. In such instances, the software 641 can determine collision avoidance with respect to each robotic manipulator 620 and each surgical tool 610 and further determine location and dimensional data relative to each robotic manipulator 620 and each surgical tool 610 within a surgical operation coordinate system. In this manner, software 641 can further identify potential collision with each robotic manipulator 620 and each surgical tool 610 to the patient and further identify potential collision with other surgical tools and robotic manipulators operating within the surgical operation coordinate system. Similarly to that described above, the software 641 can perform collision avoidance routines in real-time such that each surgical tool 610 and each robotic manipulator 620 are continuously being monitored to avoid potential collisions.

Furthermore, the software 641 can include a calibration module in order to calibrate the surgical tool 610 and the robotic manipulator 620. With respect to calibration, software 641 can pull tool data 613 from data 642 that was previously retrieved from the tool interface 643 from connector 612; however, in certain embodiments, software 641 can invoke the tool interface 643 to retrieve tool data 613 via connector 612 if the tool data 613 is not currently residing in data 642. Additionally, software 641 can pull data related to the robotic manipulator 620 from data 642. In certain embodiments, a plurality of surgical tools 610 can be utilized, as such, the software module can pull tool data 613 for each of the plurality of surgical tools 610. In some embodiments, a plurality of robotic manipulators 620 can be utilized, as such, the software module can pull data related to each robotic manipulator 620 from data 642.

In general, when determining calibration data for the surgical tool 610 and the robotic manipulator 620, software 641 can utilize actuation pressures, current/voltage requirements, optical parameters, sensor data, manufacturing data (e.g., slope of pneumatic actuation curves for cutting determined during testing by the manufacturer), signal connection data, and combinations of the same and like. In this manner, the software 641 can retrieve data related to a specific surgical tool 610 needed for operation and store tool-specific data needed for configuring the robotic manipulator 620 and/or the surgical tool 610 to be used by the control system 630. Calibration data can be stored in data 642 or other portions of memory 632. In certain embodiments, software 641 can subsequently communicate calibration data to the robotic manipulator 620 via the robotic manipulator interface 644. This can allow, for example, the robotic manipulator interface 644 to apply correct pneumatic pressure for actuation of surgical tool 610, apply correct current/voltage to operate the surgical tool 610, and combinations of the same and like. Additionally, in some embodiments, the software 641 can store the calibration data within data 642 such that any component of the robotic surgical system 600 can access and use the calibration data according to standard operating procedures for the surgical tool 610. For example, control of suction lines, fibers for lasers and endo-illumination, and wires for radio frequency (diathermy) can also be included in calibration data.

In some embodiments, the tool interface 643 can retrieve tool data 613 from the surgical tool 610 that can include, for example, mass-related data, dimensional data, and calibration data, as described above, and further collect tool identification data. In certain embodiments, tool identification data can include, for example, a serial number of the surgical tool 610, a description of the surgical tool 610, a name of the surgical tool 610, and combinations of the same and like. In some embodiments, the tool data 613 can include length of the surgical tool 610 relative to the working end, an offset of the surgical tool 610, an external profile of the surgical tool 610, operation parameters of the surgical tool 610, and combinations of the same and like.

In some embodiments, utilizing identification data from the surgical tool 610 can allow for other types of data to be determined via retrieval from a database stored in memory 632 (e.g., in data 642). For example, in some embodiments, the tool interface 643 can receive tool data 613 that includes tool identification data. After retrieval of the tool identification data, the software 641 can then retrieve mass-related data, dimensional data, and calibration data, as described above, from a database within data 642 or any portion of memory 632. This allows the software 641 to perform the various functionality as described above (e.g., gravitation compensation, collision avoidance, and/or calibration routines). As such, this allows for control system 630 to control the robotic manipulator 620 and surgical tool 610 based on the mass-related data, dimensional data, and calibration data as determined from the tool identification data (e.g., a serial number, a description, and/or a name of the surgical tool 610) stored in tool data 613. In certain embodiments, a plurality of surgical tools 610 can be utilized, as such, the software module can pull tool data 613 for each of the plurality of surgical tools 610.

As shown in FIG. 6, the control system 630 includes the robotic manipulator interface 644 to operate/control the robotic manipulator 620. In certain embodiments, the robotic manipulator interface 644 can communicate with the control system 630 and/or the robotic manipulator 620 via wired and/or wireless connections. In certain embodiments, the robotic manipulator interface 644 can include, without limitation, an EISA bus, an FSB, an ISA bus, an LPC bus, an MCA bus, a PCI bus, a PCIe bus, a SATA bus, a USB connection, an Ethernet interface, a Wi-Fi interface, a Bluetooth interface, a BLE Bluetooth interface, etc. While FIG. 6 shows the robotic manipulator interface 644 within the control system 630, the robot manipulator interface 644 can reside, alternatively, external to the control system 630.

In certain embodiments, the robotic manipulator interface 644 includes a driver within the robotic manipulator interface 644 and/or the robotic manipulator 620 in order to operate and/or manipulate the robotic manipulator 620 by the control system 630. In certain embodiments, the driver provides a software and/or hardware (e.g., firmware) interface between the robotic manipulator 620 and the robotic manipulator interface 644, enabling, for example, the control system 630 directly or indirectly access hardware functions of the robotic manipulator 620. In certain embodiments, the driver of the robotic manipulator interface 644 communicates with the control system 630, such that when the control system 630 invokes a routine through the robotic manipulator interface 644 to the robotic manipulator 620, the driver issues commands to the robotic manipulator 620. In some embodiments, the robotic manipulator interface 644 can provide two-way access/communication between the robotic manipulator 620 and the control system 630.

In certain embodiments, the robotic manipulator interface 644 can control optical connections/controls, pneumatic connections/controls, electrical connections/controls, data connections, power connections/controls, signal (e.g., optical) connections/controls, and the like of the robotic manipulator 620. Additionally, in certain embodiments, the robotic manipulator interface 644 can control various portions of the robotic manipulator 620, for example, specific joints and links of the robotic manipulator 620. In some embodiments, the robotic manipulator interface 644 can control external pneumatic pumps corresponding to functionality of the robotic manipulator 620.

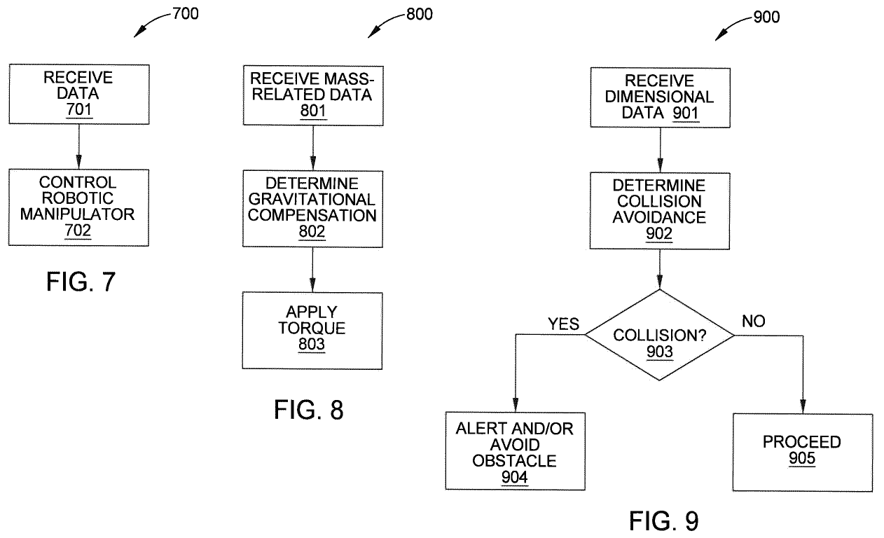

FIG. 7 illustrates a flow diagram illustrating method 700 of controlling a robotic manipulator, such as robotic manipulator 105 or robotic manipulator 620, according to certain aspects of the disclosure. In certain embodiments, the method 700 is performed by the control system 630 via the tool interface 643 by way of software 641 invoking the tool interface to transfer tool data 613 from the connector 612. At 701, data is received from a surgical tool connected to a working end of robotic manipulator 620. In some embodiments, the surgical tool can include, without limitation, the surgical tool 170 or the surgical tool 610. In some embodiments, the data can be received, for example, from the surgical tool 610 via the connector 612. In some embodiments, the data received can be, for example, tool data 613. At 702, the robotic manipulator (e.g., the robotic manipulator 620) is controlled based, at least in part, on the data received from the surgical tool. In some embodiments, the robotic manipulator is controlled by the control system 630 via the robotic manipulator interface 644 communicating with the robotic manipulator 620. In certain embodiments, the method 700 can be continually performed throughout the duration of a surgical procedure. In some embodiments, the method 700 can be performed in real-time during the surgical procedure.

FIG. 8 illustrates a flow diagram of method 800 for applying a torque value to a joint of a robotic manipulator, such as robotic manipulator 105 or robotic manipulator 620, according to certain aspects of the disclosure. In certain embodiments, the method 800 is performed by the control system 630 via the tool interface 643 by way of software 641 invoking the tool interface to transfer tool data 613 from the connector 612. At 801, mass-related data is received from a surgical tool connected to a working end of robotic manipulator 620. In some embodiments, the surgical tool can include, without limitation, the surgical tool 170 or the surgical tool 610. In some embodiments, the data can be received, for example, from the surgical tool 610 via the connector 612. In some embodiments, the data received can be, for example, tool data 613. At 802, gravitational compensation is determined by the software 641 as discussed above relative to FIG. 6. At 803, a torque value is applied to at least one joint of robotic manipulator 620 (e.g., the robotic manipulator 620) to compensate for gravitational effects of a surgical tool (e.g., the surgical tool 610) based, at least in part, on the mass-related data. In certain embodiments, 801 corresponds to 701 and 802 to 803 correspond to 702 of the method 700. In certain embodiments, the method 800 can be continually performed throughout the duration of a surgical procedure. In some embodiments, the method 800 can be performed in real-time during the surgical procedure.

FIG. 9 illustrates a flow diagram of method 900 for avoiding collision with obstacles in an operational volume of a robotic manipulator, such as robotic manipulator 105 or robotic manipulator 620, according to certain aspects of the disclosure. FIG. 9 illustrates a flow diagram of a method 900 for avoiding collision with obstacles in an operational volume of the robotic manipulator. In certain embodiments, the method 900 is performed by the control system 630 via the tool interface 643 by way of software 641 invoking the tool interface to transfer tool data 613 from the connector 612. At 901, dimensional data is received from a surgical tool connected to a working end of robotic manipulator 620. In some embodiments, the surgical tool can include, without limitation, the surgical tool 170 or the surgical tool 610. In some embodiments, the data can be received, for example, from the surgical tool 610 via the connector 612. In some embodiments, the data received can be, for example, tool data 613. At 902, collision avoidance is determined by the software 641 as discussed above relative to FIG. 6. At 903, the control system 630 identifies whether a potential collision is imminent by the software 641 as discussed above relative to FIG. 6. If a potential collision is found, the method 900 proceeds to 904. At 904, the control system 630 alerts a surgeon and/or avoids collision (e.g., automatically stopping or changing trajectory) as discussed above relative to FIG. 6. If no potential collision is found, the method 900 proceeds to 905 which includes continued operation of the surgical tool (e.g., the surgical tool 610) via the robotic manipulator (e.g., the robotic manipulator 620). In certain embodiments, 901 corresponds to 701 and 902 to 905 correspond to 702 of the method 700. In certain embodiments, the method 900 can be continually performed throughout the duration of a surgical procedure. In some embodiments, the method 900 can be performed in real-time during the surgical procedure.

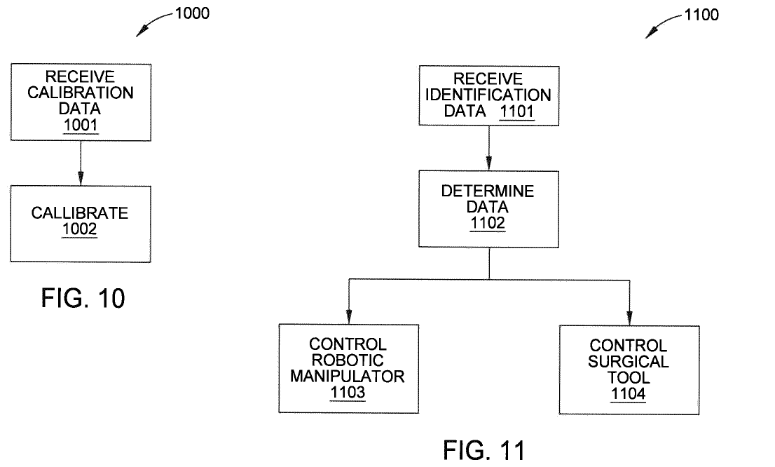

FIG. 10 illustrates a flow diagram of method 1000 for calibrating operation of a surgical tool, such as surgical tool 170 or surgical tool 610, according to certain aspects of the disclosure. In certain embodiments, the method 1000 is performed by the control system 630 via the tool interface 643 by way of software 641 invoking the tool interface to transfer tool data 613 from the connector 612. At 1001, calibration data is received from a surgical tool connected to a working end of robotic manipulator 620. In some embodiments, the surgical tool can include, without limitation, the surgical tool 170 or the surgical tool 610. In some embodiments, the data can be received, for example, from the surgical tool 610 via the connector 612. In some embodiments, the data received can be, for example, tool data 613.

At 1002, calibration of the surgical tool (e.g., surgical tool 610) is performed by the software 641 as discussed above relative to FIG. 6 based, at least in part, on the calibration data. In certain embodiments, as discussed relative to FIG. 6, calibration can include calibrating, for example, the surgical tool 610 and the robotic manipulator 620 for operation of the surgical tool 610 by the robotic manipulator 620. In certain embodiments, 1001 corresponds to 701 and 1002 corresponds to 702 of the method 700. In some embodiments, 1002 can occur before and/or during controlling the robotic manipulator (e.g., before and/or during 702 of the method 700). In certain embodiments, the method 1000 can be continually performed throughout the duration of a surgical procedure. In some embodiments, the method 1000 can be performed in real-time during the surgical procedure.

FIG. 11 illustrates a flow diagram of method 1100 for controlling surgical tool 610 and robotic manipulator 105 or robotic manipulator 620 based on identification data of the surgical tool, according to certain aspects of the disclosure. In certain embodiments, the method 1100 is performed by the control system 630 via the tool interface 643 by way of software 641 invoking the tool interface to transfer tool data 613 from the connector 612. At 1101, identification data is received. In general, identification data can include information such as, but not limited to, a serial number of the surgical tool, a description of the surgical tool, a name of the surgical tool, and combinations of the same and like. At 1102, appropriate data corresponding to the tool (e.g., surgical tool 610) is determined. As discussed above relative to FIG. 6, identification data can be used to query a database in, for example, the data 642. From this database, control system 630, for example, can identify relevant data including, but not limited to, mass-related data, dimensional data, and calibration data of the surgical tool based on the identification data of the surgical tool. At 1103, the robotic manipulator can be controlled based, at least in part, on the mass-related data of the surgical tool and the dimensional data of the surgical tool in any manner as described above relative to FIG. 6. At 1104, the operation of the surgical tool is controlled based, at least in part, on the calibration data of the surgical tool in any manner as described above relative to FIG. 6. As illustrated in FIGS. 11, 1103 and 1104 can be performed in parallel. In other embodiments, 1103 and 1104 can be conducted in sequence/tandem. In certain embodiments, the controlling of the robotic manipulator and the surgical tool can utilize software 641 to perform gravitation compensation, collision avoidance, and calibration routines as described above relative to FIG. 6. In certain embodiments, 1101 corresponds to 701 and 1102 to 1104 correspond to 702 of the method 700. In certain embodiments, the method 1100 can be continually performed throughout the duration of a surgical procedure. In some embodiments, the method 1100 can be performed in real-time during the surgical procedure.

In certain embodiments, FIGS. 8-11 show variations of the embodiment illustrated in FIG. 7. It should be noted that, in some embodiments, each of the methods 700, 800, 900, 1000, and 1100 can be performed in parallel or in tandem. Furthermore, in certain embodiments, the methods 700, 800, 900, 1000, and 1100 correspond to processes performed by the control system 630 via software 641 invoking the tool interface 643 and/or the robotic manipulator interface 644 to communicate with the connector 612 and/or the robotic manipulator 620, respectively, as described above relative to FIG. 6. In certain embodiments, methods 700, 800, 900, 1000, and 1100 can be performed continuously and/or in real-time while a surgical operation is taking place. In some embodiments, methods 700, 800, 900, 1000, and 1100 can be performed automatically when a surgical tool is connected to the robotic manipulator.

In one embodiment, a system includes a robotic manipulator with a working end coupled to a base by a plurality of joints and a plurality of links, a surgical tool, and a control system. The surgical tool includes a tool portion and a connector portion coupled to the working end of the robotic manipulator. The control system includes a tool interface operable to communicate with the surgical tool, a robotic manipulator interface communicatively coupled to the robotic manipulator, and a processor coupled to the tool interface and the robotic manipulator interface. The processor is configured to receive data from the surgical tool and control the robotic manipulator based, at least in part, on the data received from the surgical tool.

In another embodiment of the system, the tool portion includes a phacoemulsification tool, a vitrectomy tool, a laser tool, or an optical tool.

In another embodiment of the system, the data received from the surgical tool includes mass-related data of the surgical tool, and the control includes applying a torque value to at least one joint to compensate for gravitational effects of the surgical tool based, at least in part, on the mass-related data of the surgical tool.

In another embodiment of the system, the data received from the surgical tool includes dimensional data of the surgical tool, and the control includes avoiding collision with obstacles in an operational volume of the robotic manipulator based, at least in part, on the dimensional data of the surgical tool.

In another embodiment of the system, the data received from the surgical tool includes calibration data of the surgical tool and the processor is further configured to calibrate operation of the surgical tool based, at least in part, on the calibration data of the surgical tool.

In another embodiment of the system, the data received from the surgical tool includes identification data, and the control includes determining mass-related data, dimensional data and calibration data of the surgical tool based on the identification data of the surgical tool, controlling the robotic manipulator based, at least in part, on the mass-related data of the surgical tool and the dimensional data of the surgical tool, and controlling operation of the surgical tool based, at least in part, on the calibration data of the surgical tool.

In another embodiment of the system, the control system is operable to communicate with the tool interface via a wireless connection or a wired connection.

Although various embodiments of the present disclosure have been illustrated in the accompanying drawings and described in the foregoing detailed description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments are possible in which these tasks are performed by a different entity.

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

Although various embodiments of the method and apparatus of the present invention have been illustrated in the accompanying drawings and described in the foregoing Detailed Description, it will be understood that the invention is not limited to the embodiments disclosed, but is capable of numerous rearrangements, modifications and substitutions without departing from the spirit of the invention as set forth herein.

What is claimed is:

1. A method for performing robotic surgery, comprising:
receiving data from a surgical tool connected to a working end of a robotic manipulator; and
controlling the robotic manipulator based, at least in part, on the data received from the surgical tool.

2. The method of claim 1, wherein:
the data received from the surgical tool comprises mass-related data of the surgical tool; and
the controlling comprises applying a torque value to at least one joint to compensate for gravitational effects of the surgical tool based, at least in part, on the mass-related data of the surgical tool.

3. The method of claim 2, wherein the mass-related data of the surgical tool comprises:
a mass;
a center of mass location defined with respect to a surgical tool coordinate system;
a moment of inertia; and
a length of the surgical tool.

4. The method of claim 1, wherein:
the data received from the surgical tool comprises dimensional data of the surgical tool; and
the controlling comprises avoiding collision with obstacles in an operational volume of the robotic manipulator based, at least in part, on the dimensional data of the surgical tool.

5. The method of claim 4, wherein the dimensional data of the surgical tool comprises:
a length of the surgical tool;
a first maximum offset from a centerline of the surgical tool in a first dimension;
a second maximum offset from the centerline of the surgical tool in a second dimension; and
a third maximum offset from the centerline of the surgical tool in a third dimension.

6. The method of claim 1, wherein:
the data related to the surgical tool comprises calibration data of the surgical tool; and
the method further comprises calibrating operation of the surgical tool based, at least in part, on the calibration data of the surgical tool.

7. The method of claim 6, wherein the calibration data comprises at least one of mass-related data of the surgical tool, dimensional data of the surgical tool, data needed to operate the surgical tool, power requirements of the surgical tool, pressures required for pneumatic operations of the surgical tool, light wavelengths required by the surgical tool, or light strengths required by the surgical tool.

8. The method of claim 1, wherein:
the data related to the surgical tool comprises identification data; and
the controlling comprises:
determining mass-related data, dimensional data and calibration data of the surgical tool based on the identification data of the surgical tool;
controlling the robotic manipulator based, at least in part, on the mass-related data of the surgical tool and the dimensional data of the surgical tool; and
controlling operation of the surgical tool based, at least in part, on the calibration data of the surgical tool.

9. The method of claim 8, wherein the identification data related to the surgical tool comprises:
a serial number of the surgical tool;
a description of the surgical tool; and
a name of the surgical tool.

10. The method of claim 1, wherein:
the surgical tool comprises a tool portion and a connector portion; and
the tool portion comprises a phacoemulsification tool, a vitrectomy tool, a laser tool, or an optical tool.

11. A surgical tool, comprising:
a tool portion; and
a connector portion configured to be coupled to a working end of a robotic manipulator, the connector portion comprising:
a communication interface; and
a controller, coupled to the communication interface, configured to send data to a tool interface of a robotic manipulator control system.

12. The surgical tool of claim 11, wherein the tool portion comprises a phacoemulsification tool, a vitrectomy tool, a laser tool, or an optical tool.

13. The surgical tool of claim 11, wherein:
a proximal end of the connector portion comprises a tool connection interface configured to align and connect to a corresponding robotic manipulator connection interface of the working end;
the tool connection interface comprises an optical connection, a pneumatic connection, an electrical connection, a data connection, a power connection, or a signal connection;
the communication interface is operable to communicate with the tool interface via a wireless connection or a wired connection; and
the connector portion stores data corresponding to the surgical tool.
